(19) United States
(12) Patent Application Publication
(10) Pub. No.: US 2011/0003280 A1
(43) Pub. Date: Jan. 6, 2011

(54) IMMUNOASSAY APPARATUS AND IMMUNOASSAY METHOD

(75) Inventors: Youichi Takahama, Miki-shi (JP); Takashi Kagawa, Kobe-shi (JP); Shinya Nagai, Akashi-shi (JP); Miki Miyaji, Kako-gun (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATRION

(21) Appl. No.: 12/920,101

(22) PCT Filed: Feb. 8, 2009
(86) PCT No.: PCT/JP2009/052788

§ 371 Date: Aug. 27, 2010

(30) Foreign Application Priority Data
Feb. 29, 2008 (JP) 2008-050300
Feb. 29, 2008 (JP) 2008-050329
Feb. 29, 2008 (JP) 2008-050370

(51) Int. Cl. C12Q 1/70; C12M 1/34

(52) U.S. Cl. 435/5; 435/287.2

(57) ABSTRACT

The present invention relates to an immunoassay apparatus comprising a measurement unit 2 for measuring a HIV antibody amount and a HIV antigen amount in a specimen and an analysis means 400 for analyzing on HIV infection based on a measurement result which is outputted from the measurement unit 2. The analysis means 400 includes a false-positive detection means for detecting false-positive as a result of a HIV infection test, and the false-positive detection means detects false-positive when the HIV antibody amount is within a range of weak-positive and the HIV antigen amount is within a range of negative.

### TECHNICAL FIELD

The present invention relates to an immunoassay apparatus and an immunoassay method which are used for a HIV infection test.

### BACKGROUND ART

When a patient is infected with a HIV virus, an amount of HIV antigen increases in the patient body in an early stage. Subsequently, an amount of HIV antibody increases while the HIV antigen gradually decreases or disappears due to the antigen-antibody reaction in the body. Further subsequently, the HIV antigen proliferates again due to a depressed immune apparatus.

In a HIV test for testing such HIV infection, a HIV antibody test has commonly prevailed.

However, in the HIV antibody test, there is a so-called window stage for a period of one to three months after the infection when a test result is negative because the antibody does not increase even though the patient is infected with HIV. This has been a problem.

Further, in published Japanese translation of a PCT application No. 2001-504572 and published Japanese translation of a PCT application No. 2001-514749, a reagent of antigen-antibody simultaneous detection for simultaneously measuring the HIV antigen and the HIV antibody is disclosed.

Further, it is known that an antigen HIV-1 p24 becomes positive in an early infection stage before an antibody to HIV is produced and in an AIDS stage. Therefore, it is attempted to shorten the window stage where the test result is negative even though the patient is infected with HIV, by measuring the antigen HIV-1 p24 using the above-described reagent of the antigen-antibody simultaneous detection.

However, although it is possible that the above-described reagent of antigen-antibody simultaneous detection shortens the window stage, there is another problem that “false positive” is often determined as positive instead of negative. This is attributed to measurement precision of the antibody for example, and the antibody may be determined as positive due to nonspecific reaction when the antibody is measured.

In the case where the false positive occurs in the HIV test, a mental stress on the subject is extremely heavy.

It is also important to figure out a course degree (typically a so-called clinical stage) or how long time has passed since the specimen is infected with HIV, for doctors deciding on a policy of treatment/test for patients.

However, in the immunoassay apparatus using the antigen-antibody simultaneous detection reagent as described above, it has been impossible to determine a course degree, or how long time has passed since the specimen is infected with HIV, though it has been possible to determine whether the specimen is positive or negative.

Further, in the antigen-antibody simultaneous detection reagent as described above, it is impossible to judge whether a HIV antigen is positive or a HIV antibody is positive and it is hard to determine a disease state of the subject, in a case where the result is determined as positive.

### SUMMARY OF INVENTION

Therefore, one of objectives of the present invention is to provide an immunoassay apparatus and an immunoassay method which can decrease determination of false-positive as positive.

Another objective of the present invention is to provide an immunoassay apparatus which can determine a course degree since the HIV infection.

Another objective of the present invention is to provide an immunoassay apparatus which can output respective analysis results of HIV antigen and HIV antibody, besides presence of HIV infection.

According to the first aspect of the present invention, an immunoassay apparatus includes a measurement unit for measuring a HIV antibody amount and a HIV antigen amount which are included in a specimen and an analysis means for analyzing on HIV infection based on a measurement result which is outputted from the measurement unit, wherein the analysis means includes a false-positive detection means for detecting false-positive as a result of a HIV infection test and the detection means detects false-positive when the HIV antibody amount is within a range of weak-positive and the HIV antigen amount is within a range of negative.

It is preferable to further include an output means for producing an output indicating that determination of the HIV infection test is suspended or that the HIV infection test is resulted as negative in a case where the false-positive is detected.

The measurement unit preferably includes an antibody measurement means for measuring the HIV antibody amount in a first aliquot of the specimen and an antigen measurement means for measuring the HIV antigen amount in a second aliquot of the specimen.

The measurement unit preferably measures the HIV antibody amount and the HIV antigen amount in parallel.

The measurement unit preferably measures the HIV antibody amount and the HIV antigen amount by measuring a chemiluminescence amount of the specimen.

It is preferable to further include a message output means for outputting a message prompting a retest in a case where false-positive is detected.

The analysis means preferably analyzes presence or absence of HIV infection and a course degree since HIV infection.

The HIV antigen is preferably an antigen HIV-1 p24.

The HIV antibody is preferably anti-envelope antibody, anti-core antibody, or anti-polymerase antibody.

The HIV antibody is preferably IgG antibody, IgA antibody, or IgM antibody.

According to another aspect of the present invention, an immunoassay method includes an antibody measurement step of measuring a HIV antibody amount in a specimen, an antigen measurement step of measuring a HIV antigen amount in the specimen, and an analysis step of analyzing on HIV infection based on a measurement result which is obtained in the antibody measurement step, wherein the analysis step includes a false-positive detection step of detecting false-positive as a result of a HIV infection test, and in the false-positive detection step, false-positive is detected when the HIV antibody amount is within a range of weak-positive and the HIV antigen amount is within a range of negative.

It is preferable to further include an output step of producing an output indicating that determination of HIV infection test is suspended or that HIV infection test is resulted as negative in a case where the false-positive is detected.

It is preferable to further include a first aliquot obtaining step of obtaining a first aliquot from the specimen and a second aliquot obtaining step of obtaining a second aliquot from the specimen, and a HIV antibody amount which is included in the first aliquot is measured in the antibody measurement step, and a HIV antigen amount which is included in the second aliquot is measured in the antigen measurement step.

According to another aspect of the present invention, an immunoassay apparatus includes a measurement unit for measuring a HIV antibody amount and a HIV antigen amount in a specimen and an analysis unit for analyzing on HIV infection based on a measurement result which is outputted from the measurement unit and for detecting false-positive as a result of a HIV infection test when the HIV antibody amount is within a range of weak-positive and the HIV antigen amount is within a range of negative.

It is preferable to further include a display to produce an output indicating that determination of HIV infection test is suspended or that HIV infection test is resulted as negative in a case where the false-positive is detected.

According to another aspect of the present invention, an immunoassay apparatus includes a measurement unit for measuring a HIV antibody amount and a HIV antigen amount in a specimen and an analysis unit for analyzing a course degree since HIV infection based on a measurement result outputted from the measurement unit.

The analysis unit preferably determines whether or not the specimen is one in an early infection stage based on the measurement result outputted from the measurement unit.

The analysis unit preferably determines whether or not the specimen is one in an AIDS stage based on the measurement result outputted from the measurement unit.

The analysis unit preferably determines whether the specimen is in the early infection stage, in an asymptomatic stage following the early inspection stage, or in an AIDS stage following the asymptomatic stage based on the measurement result outputted from the measurement unit.

The analysis unit preferably determines whether or not the specimen is positive based on the measurement result outputted from the measurement unit and analyzes a course degree since the HIV infection in a case where the specimen is determined as positive.

The analysis unit preferably determines that the specimen is one in the early infection stage when a HIV antigen amount measured by the measurement unit is an amount indicative of positive and a HIV antibody amount measured by the measurement unit is less than a first threshold.

The analysis unit preferably determines that the specimen is one in the AIDS stage when the HIV antigen amount measured by the measurement unit is an amount indicative of positive and the HIV antibody amount measured by the measurement unit is an amount indicative of positive and is larger than a second threshold.

The analysis unit preferably determines that the specimen is one in the asymptomatic stage when the HIV antigen amount measured by the measurement unit is an amount indicative of negative and the HIV antibody amount measured by the measurement unit is an amount indicative of positive.

According to other aspect of the present invention, an immunoassay apparatus includes a measurement unit for measuring a HIV antibody and a HIV antigen which are included in a specimen, an antibody analysis means for analyzing a HIV antibody measurement result by the measurement unit, an antigen analysis means for analyzing a HIV antigen measurement result by the measurement unit, an infection determination means for determining presence or absence of HIV infection based on integration of the HIV antibody measurement result and the HIV antigen measurement result which are measured by the measurement unit, and an output means for outputting the determination result by the infection determination means, the analysis result made by the antibody analysis means and the analysis result made by the antigen analysis means.

It is preferable that the antibody analysis means generates antibody amount information indicative of the HIV antibody amount, the antigen analysis means generates antigen amount information indicative of a HIV antigen amount, and the output means outputs the antibody amount information as an analysis result by the antibody analysis means and outputs the antigen amount information as an analysis result by the antigen analysis means.

It is preferable to configure so that the antibody analysis means determines whether the HIV antibody is positive or negative based on the HIV antibody measurement result by the measurement unit, the antigen analysis means determines whether the HIV antigen is positive or negative based on the HIV antigen measurement result by the measurement unit, and the output means outputs the HIV antibody determination result as an analysis result by the antibody analysis means and the HIV antigen determination result as an analysis result by the antigen analysis means.

The infection determination means preferably determines presence or absence of HIV infection using the HIV antibody determination result and the HIV antigen determination result.

The infection determination means preferably determines that HIV infection presents in a case where the HIV antibody measurement result by the measurement unit indicates HIV antibody positive and/or the HIV antigen measurement result by the measurement unit indicates HIV antigen positive.

It is preferable to further include a transportation device for transporting the specimen container to a suction position for the measurement unit to suck in the specimen.

The output means preferably chronologically displays a determination result by the infection determination means, an analysis result by the antibody analysis means, and an analysis result by the antigen analysis means with respect to a specific subject.

According to another aspect of the present invention, an immunoassay apparatus includes: a measurement unit for measuring a HIV antibody and a HIV antigen which are included in a specimen; an analysis unit for analyzing a HIV antibody measurement result by the measurement unit and a HIV antigen measurement result by the measurement unit, and determining presence or absence of HIV infection based on integration of the HIV antibody measurement result and the HIV antigen measurement result which are measured by the measurement unit; and a display for outputting an analysis result of the HIV antigen measurement result, an analysis result of the HIV antibody measurement result, and a HIV infection determination result.

### BRIEF DESCRIPTION OF DRAWINGS

FIG. 1 is an overall view showing an immunoassay apparatus.

FIG. 2 is a detailed view showing a measurement unit.

FIG. 3 is a configuration view showing a control device.

FIG. 4 is a flowchart of a HIV assay process.

FIG. 5 is a flowchart of HIV antibody measurement process and HIV antigen measurement process.

FIG. 6 is a principle view showing HIV antibody measurement.

FIG. 7 is a principle view showing HIV antigen measurement.

FIG. 8 is a flowchart of an analysis process.

FIG. 9 is a view showing a relation between a chronological change of antibody amount and antigen amount and a clinical stage.

FIG. 10 is a flowchart showing a nonspecific reaction determination process.

FIG. 11 is a view showing a range of antibody weak-positive in the relation view between the chronological change of the antibody amount and antigen amount and the clinical stage.

FIG. 12 is a flowchart of a determination process on negative, positive and course degree since infection.

FIG. 13 is an example of display screen of a determination result.

FIG. 14 is a view showing a measurement result.

FIG. 15 is a view showing a measurement result.

FIG. 16 is a view showing a measurement result.

FIG. 17 is a view showing a chronological display screen.

FIG. 18 is a principle view showing another example of HIV antibody measurement.

### DESCRIPTION OF EMBODIMENTS

Hereinafter, embodiments of a sample assay apparatus of the present invention are explained in detail with reference to figures attached hereto.

### [Overall Configuration of Apparatus]

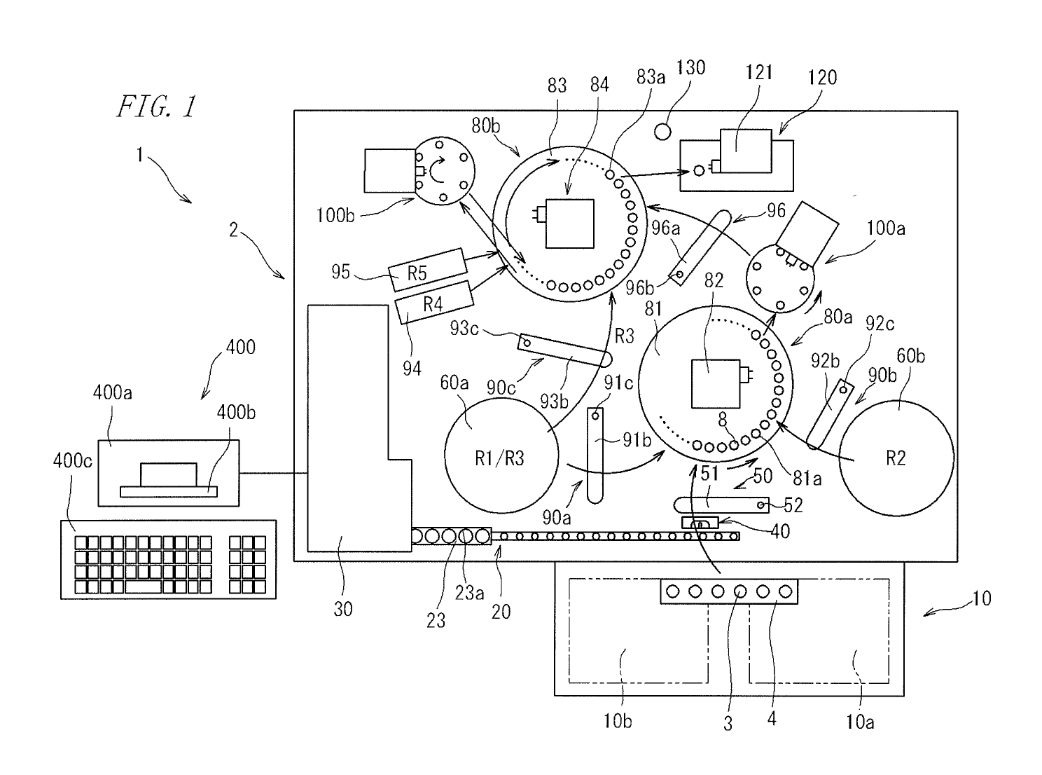

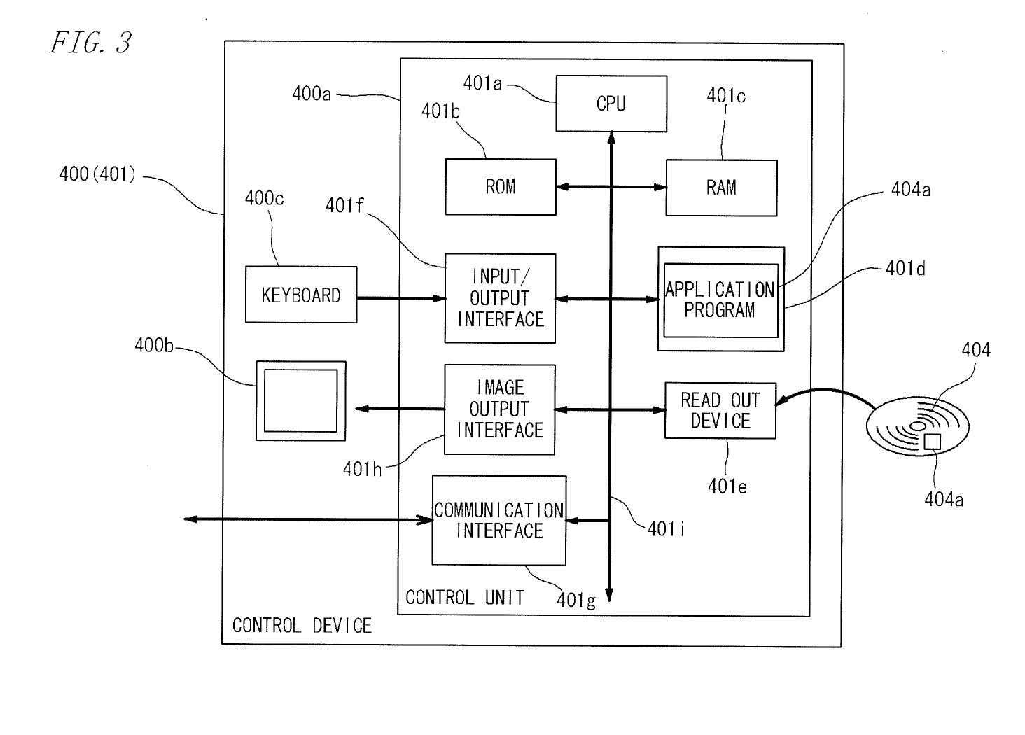

FIG. 1 is an explanatory plan view showing an overall configuration of an immunoassay apparatus (sample assay apparatus) according to an embodiment of the present invention. The immunoassay apparatus 1 according to the embodiment of the present invention is an apparatus for testing HIV infection using a sample (specimen) such as blood. As shown in FIG. 1, this immunoassay apparatus 1 is primarily configured by a measurement unit 2 having a plurality of mechanisms (components) and a control device 400 being a data process unit which is electrically connected to the measurement unit 2 (ref. to FIG. 3).

The measurement unit 2 has a function of measuring a HIV antibody in a first aliquot of a specimen and a function of measuring a HIV antigen in a second aliquot of the specimen. Therefore, in the measurement unit 2, the same specimen is measured respectively by the function of measuring the HIV antibody and the function of measuring the HIV antigen.

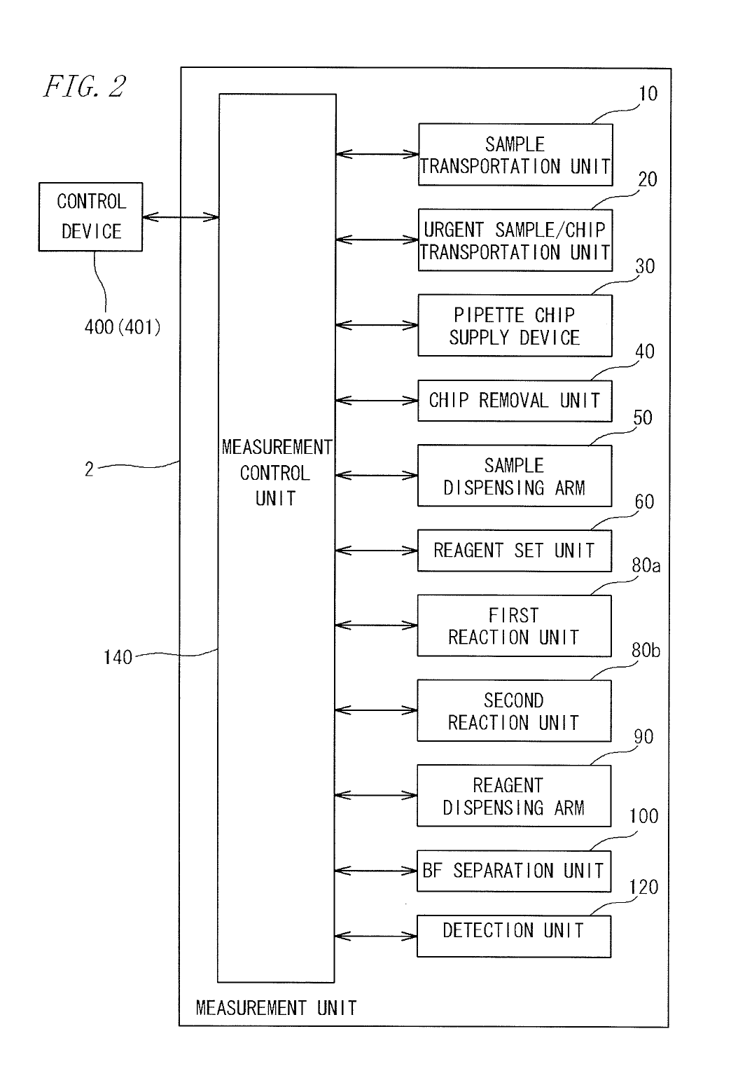

As shown in FIG. 2, this measurement unit 2 includes a sample transportation unit (sampler) 10; an urgent sample/chip transportation unit 20; a pipette chip supply unit 30; a chip removal unit 40; a sample dispensing arm 50; reagent set units 60a and 60b; a first reaction unit 80a and a second reaction unit 80b; reagent dispensing arms 90a, 90b, and 90c; a first B/F separation unit 100a and a second B/F separation unit 100b; a detection unit 120; and a measurement control unit 140 for controlling operation of respective mechanisms such as the sample transportation unit (sampler) 10 and the sample dispensing arm 50 which are included in the measurement unit 2. Here, in the immunoassay apparatus 1 according to the present embodiment, a disposable pipette chip is exchanged every time the sample is suctioned and discharged in order that the sample such as blood which is suctioned and discharged by the sample dispensing arm 50 is prevented from mixing with other samples.

### [Configuration of Control Device]

The control device 400 consists of a personal computer 401 (PC) and others and includes a control unit 400a, a display unit (output means) 400b, and an input unit (input means) 400c such as a keyboard and a mouse as shown in FIG. 1. The control unit 400a controls operation of respective mechanisms in the measurement unit 2 and has a function for assaying optical information of the sample which is obtained in the measurement unit 2. This control unit 400a consists of CPU, ROM, RAM and others. Further, the display unit 400b is used for displaying information including assay results obtained in the control unit 400a.

Next, a configuration of the control device 400 is explained. As shown in FIG. 3, the control unit 400a is primarily configured by a CPU 401a, a memory unit consisting of ROM 401b, RAM 401c, a hard disk 401d, and others, a readout device 401e, an input/output interface 401f, a communication interface 401g, and an image output interface 401h. The CPU 401a, ROM 401b, RAM 401c, the hard disk 401d, the readout device 401e, the input/output interface 401f, the communication interface 401g, and the image output interface 401h are connected through a bus 401i.

The CPU 401a is capable of executing a computer program memorized by the ROM 401b and a computer program loaded on the RAM 401c. The computer 401 functions as the control device 400 by causing the CPU 401a to execute an application program 404a described later.

The ROM 401b consists of a mask ROM, PROM, EPROM, EEPROM and others and records a computer program executed by the CPU 401a, data used for this, and others.

The RAM 401c consists of SRAM, DRAM, or others. The RAM 401c is used for reading out a computer program recorded in the ROM 401b and the hard disk 401d. Further, the RAM 401c is used as a work area of the CPU 401a when these computer programs are executed.

Various computer programs 404a for causing the CPU 401a to execute such as operating systems and application programs and data used for executing the computer program are installed in the hard disk 401d. For example, an application program for registering a measurement order and an application program for calculating measurement frequency and displaying the calculation result are also installed in the hard disk 401d.

The readout device 401e consists of a flexible disk drive, a CD-ROM drive, a DVD-ROM drive, or the other and is capable of reading out a computer program or data which is recorded in a portable recording medium 404. Further, the application program 404a of the present embodiment is stored in the portable recording medium 404. The computer 401 is capable of reading out the application program 404a from the portable recording medium 404 and capable of installing the application program 404a in the hard disk 401d.

Here, the application program 404a is not only provided by the portable recording medium 404 but also may be provided by external devices which are communicably connected with the computer 401 by an electric communication line (wired or wireless), through the electric communication line. For example, it is also possible that the application program 404a is stored in a hard disk of a server computer on the Internet, the computer 401 accesses the server computer for downloading the application program 404a, and the application program 404a is installed in the hard disk 401d.

For example, an operating system which provides a graphical user interface environment such as Windows (registered trademark) which is manufactured and sold by US Microsoft Co. is installed in the hard disk 401d. In explanation given below, the application program 404a in the present embodiment is to be operated on the above-described operating system.

The input/output interface 401f consists of, for example, a serial interface such as USB, IEEE1394, and RS-232C, a parallel interface such as SCSI, IDE, IEEE1284, an analog interface consisting of a D/A converter and an A/D converter, and others. A keyboard 400c is connected to the input/output interface 401f so that the user can input data in the computer 401 by using the keyboard 400c.

The communication interface 401g is, for example, Ethernet (registered trademark) interface. The computer 401 is capable of sending and receiving data with the measurement unit 2 using a given communication protocol by the communication interface 401g.

The image output interface 401h is connected to the display unit 400b consisting of LCD, CRT, or the other and outputs an image signal depending on image data provided by the CPU 401a to the display unit 400b. The display unit 400b displays images (screen) according to the inputted image signal.

### [Configuration of Respective Mechanisms of Immunoassay Apparatus]

Conventional configurations may be applied for configurations of respective mechanisms of the immunoassay apparatus 1. They are explained briefly hereinafter.

The sample transportation unit 10 is configured so as to transport a rack 4, on which a plurality of test tubes 3 containing samples are placed, to a position corresponding to a suction position of the sample dispensing arm 50. Because the transportation device for transporting the test tube 3 (specimen container) to a suction position is provided and the test tube 3 is automatically transported by the transportation device, it is possible to reduce a risk of HIV infection of the device user.

This sample transportation unit 10 has a rack set unit 10a for setting the rack 4 on which the test tube 3 containing an unprocessed sample is placed and a rack storage unit 10b for storing the rack 4 on which the test tube 3 containing an already dispensed sample is placed. The test tube 3 containing the unprocessed sample is transported to a position corresponding to a suction position of the sample dispensing arm 50 so that samples such as blood inside the test tube 3 are suctioned by the sample dispensing arm 50 and the rack 4 having the test tube 3 placed on is stored in the rack storage unit 10b.

The urgent sample/chip transportation unit 20 is configured so that the test tube 3 containing an urgent sample which is required to cut in front of samples transported by the sample transportation unit 10 is transported to a mounting position of the sample dispensing arm 50.

The pipette chip supply unit 30 has a function of placing a fed pipette chip one by one to a chip set unit 23a of a transportation rack 23 of the urgent sample/chip transportation unit 20.

The chip removal unit 40 is provided for removing the pipette chip mounted on the sample dispensing arm 50 described later.

The sample dispensing arm 50 has a function of dispensing the sample inside the test tube 3, which is transported to a suction position by the sample transportation unit 10, into a cuvette 8 which is held by a hold unit 81a of a turn table unit 81 of the first reaction unit 80a described later. The sample dispensing arm 50 dispenses the same sample to a first cuvette 8 as a first aliquot for antibody measurement and dispenses to a second cuvette 8 as a second aliquot for antigen measurement of the sample.

This sample dispensing arm 50 is configured so as to enable the arm unit 51 to rotate around a shaft 52 as a center and move in an up-and-down direction (Z direction). A nozzle unit for suctioning and discharging the sample is provided at an end portion of the arm unit 51, and a pipette chip to be transported by a transportation rack (not shown) of the urgent sample/chip transportation unit 20 is mounted at the end of the nozzle unit.

A reagent container containing a reagent R1 and a reagent container containing a reagent R3 are set in the reagent set unit 60a.

On the other hand, a reagent container containing a reagent R2 is set in the reagent set unit 60b.

Here, in the reagent set units 60a and 60b, reagent containers containing reagents R1 to R3 for antibody measurement and reagent containers containing reagents R1 to R3 for antigen measurement are set up.

The first reaction unit 80a is provided for rotating and transporting the cuvette 8 held by the hold unit 81a of the turn table unit 81 by a predetermined angle in every predetermined period and for agitating the sample and the reagents R1 and R2 in the cuvette 8.

This first reaction unit 80a is configured with the turn table unit 81 for transporting the cuvette containing the sample and reagents R1 and R2 in a rotation direction and a container transportation unit 82 for agitating the sample and reagents R1 and R2 in the cuvette 8 and transporting the cuvette 8 containing the sample and the reagents R1 and R2 which are thus agitated to a first B/F separation unit 100a described later.

The container transportation unit 82 is rotatably set in a center of the turn table unit 81. This container transportation unit 82 has a function of grasping the cuvette which is held by the hold unit 81a of the turn table unit 81 and agitating the specimen inside the cuvette 8. Further, the container transportation unit 82 also has a function of transporting the cuvette 8 wherein the sample and the reagents R1 and R2 which are agitated and incubated are contained into the first B/F separation unit 100a.

The reagent dispensing arm 90a has a function of suctioning the reagent R1 in the reagent container set in the reagent set unit 60a and dispensing the reagent R1 thus suctioned in the cuvette of the first reaction unit 80a. This reagent dispensing arm 90a is configured so that the arm unit 91b rotates around the shaft 91c and moves in an up-and-down direction. Further, a nozzle for suctioning and discharging the reagent R1 in the reagent container is fitted at an end portion of the arm unit 91b.

The reagent dispensing arm 90b has a function of dispensing the reagent R2 in the reagent container set in the reagent set unit 60b into the cuvette 8 of the first reaction unit 80a where the sample and the reagent R1 are dispensed. This reagent dispensing arm 90b is configured so that the arm unit 92b rotates around the shaft 92c and moves in an up-and-down direction (Z direction). Further, a nozzle for suctioning and discharging the reagent R2 in the reagent container is fitted at an end portion of the arm unit 92b.

According to the present embodiment, the first B/F separation unit 100a is provided for B/F separating (washing) the specimen in the cuvette 8 which is transported by the container transportation unit 82 of the first reaction unit 80a.

The cuvette 8 of the first B/F separation unit 100a is transported to the hold unit 83a of the turn table unit 83 of the second reaction unit 80b by a transportation mechanism 96. The transportation mechanism 96 is configured so that the arm unit 96a having the cuvette grasp unit (not shown) at an end thereof rotates around the shaft 96b and moves in an up-and-down direction (z direction).

The second reaction unit 80b has a similar configuration to the first reaction unit 80a. The second reaction unit 80b is provided for rotating and moving the cuvette, which is held by the hold unit 83a of the turn table unit 83, by a predetermined angle in every predetermined period and for agitating the sample and reagents R1, R2, R3, R4, and R5 in the cuvette 8.

The second reaction unit 80b is configured by the turn table unit 83 for transporting the cuvette 8 which contains the sample and the reagents R1, R2, R3, R4, and R5 in a rotation direction and the container transportation unit 84 for agitating the sample and the reagents R1, R2, R3, R4, and R5 in the cuvette 8 and transporting the cuvette 8 containing thus agitated samples and others to the second B/F separation unit 100b described later. Further, the container transportation unit 84 has a function of transporting the cuvette 8 thus processed by the second B/F separation unit 100b to the hold unit 83a of the turn table unit 83 again.

The reagent dispensing arm 90c has a function of suctioning the reagent R3 in the reagent container set in the reagent set unit 60a and dispensing thus suctioned regent R3 into the cuvette 8 of the second reaction unit 80b where the sample and the reagents R1 and R2 are dispensed. This reagent dispensing arm 90c is configured so that the arm unit 93b rotates around the shaft 93c and moves in an un-and-down direction. Further, a nozzle for suctioning and discharging the reagent R3 in the reagent container is fitted at an end portion of the arm unit 93b.

The second B/F separation unit 100b has a similar configuration to the first B/F separation unit 100a and it is provided for separating unreacted reagent R3 (unnecessary component) and magnetic particles from the specimen in the cuvette 8 which is transported by the container transportation unit 84 of the second reaction unit 80b.

A reagent R4 supply unit 94 and a reagent R5 supply unit 95 are provided for supplying reagents R4 and R5 into the cuvette 8 which is held by the hold unit 83a of the turn table unit 83 of the second reaction unit 80b.

The detection unit 120 is provided for measuring the amount of antibody or antigen which are included in the sample, by acquiring a chemiluminescence amount from the sample (specimen) subjected to a predetermined treatment with a photo multiplier tube. The detection unit 120 has a transportation mechanism unit 121 for transporting the cuvette 8 held by the hold unit 83a of the turn table unit 83 of the second reaction unit 80b to the detection unit 120.

A used cuvette from which the specimen already measured is suctioned is discarded to a dust box not shown which is arranged under the immunoassay apparatus 1, through a discard hole 130.

Using the above configuration, the detection unit 120 outputs a chemiluminescence amount indicative of an antibody amount in a specimen (the first aliquot) and a chemiluminescence amount indicative of an antigen amount in the specimen (the second aliquot) as a measurement result.

The chemiluminescence amount indicative of an antibody amount and the chemiluminescence amount indicative of an antigen amount as a measurement result are related as a measurement result with regard to the same specimen and are provided to the control device 400.

### [Overall Process of HIV Assay]

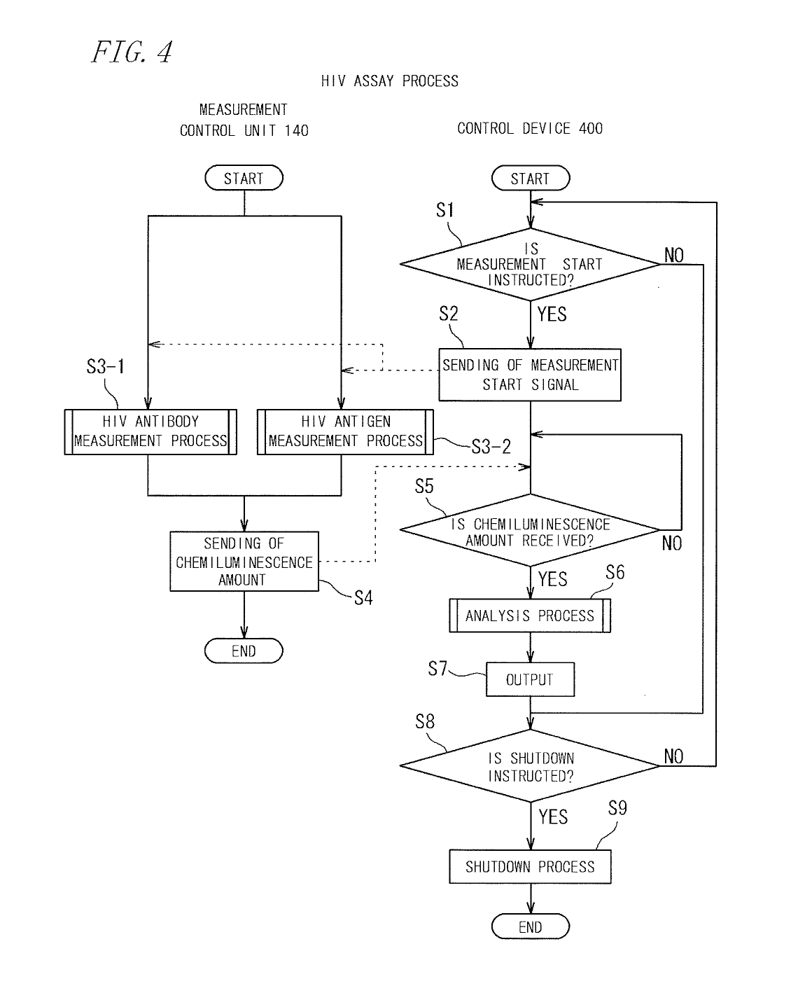

Hereinafter, HIV assay by the immunoassay apparatus 1 is explained. As shown in FIG. 4, when the control device 400 receives a measurement-start instruction (from users and others) (Step S1), the control device 400 sends a measurement-start signal to the measurement control unit 140 of the measurement unit 2 (Step S2).

When receiving the measurement-start signal, the measurement control unit 140 of the measurement unit 2 causes the measurement unit 2 to carry out specimen measurement processes (HIV antibody measurement process and HIV antigen measurement process) in parallel (Step S3-1, S3-2). It is possible to cut measurement time since the HIV antibody measurement process and the HIV antigen measurement process are carried out in parallel.

When receiving a measurement result (chemiluminescence amount) from the detection unit 120 of the measurement unit 2, the measurement control unit 140 stores the measurement result in a memory and sends it to the control device 400 (Step S4).

When receiving the measurement result (chemiluminescence amount) from the measurement control unit 140 (Step S5), the control device 400 carries out a HIV analysis process of analyzing the measurement result comprehensively (Step S6).

Then, the control device 400 outputs an analysis result (determination result on presence of HIV infection) to the display unit 400b of the control device 400 (Step S7).

Here, when receiving a shutdown instruction (Step S8), the control device 400 carries out a shutdown process (Step S9). The control device 400 may continue to measure other specimens in a case where the shutdown instruction is not provided.

### [Measurement Process]

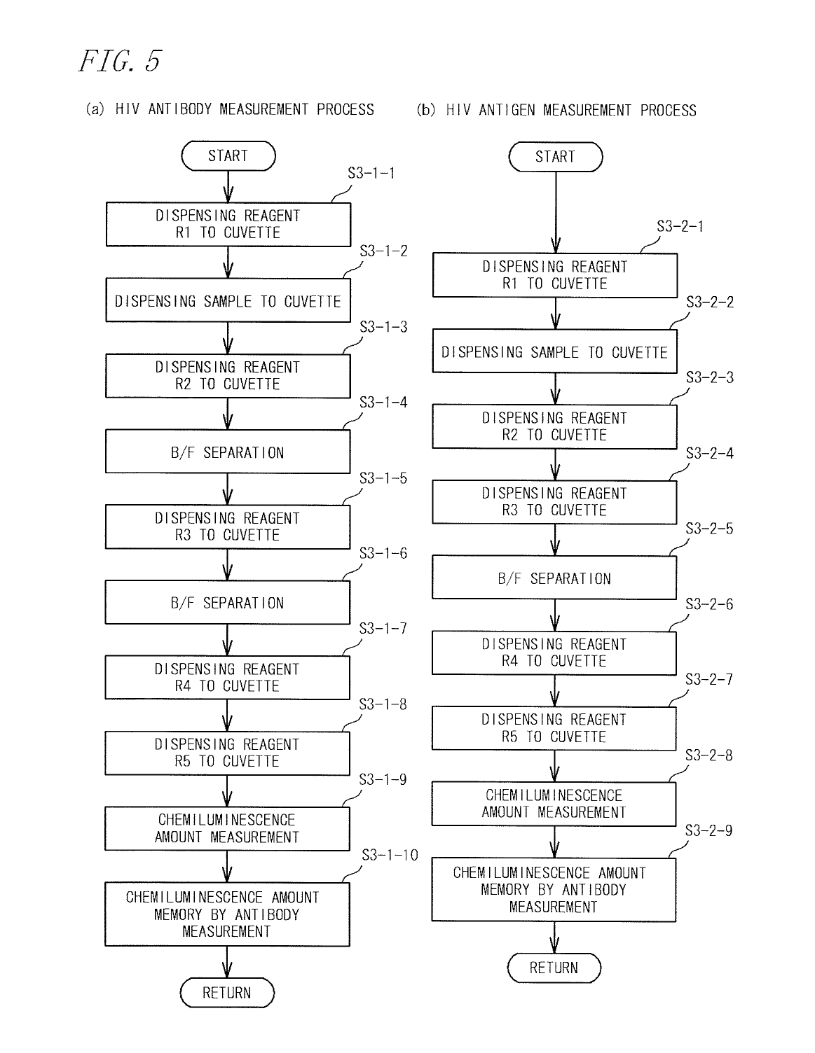

Hereinafter, a HIV antibody measurement process and a HIV antigen measurement process are explained with reference to FIGS. 5 to 7.

### [Step S3-1: HIV Antibody Measurement Process]

A HIV antibody measurement process according to the present embodiment is purposed to measure a HIV-IgG antibody (anti-envelope antibody (env.)) in the first aliquot of the specimen. Here, an antibody to be measured may be not only anti-envelop antibody (env.) but also anti-core antibody (gag.) or anti-polymerase antibody (pol.).

Further, an antibody to be measured may be IgA antibody, IgM antibody and others instead of IgG antibody.

In the antibody measurement process, as shown in FIG. 5(a), first a reagent R1 (buffer solution) for antibody measurement is dispensed in the first cuvette 8 held by the first reaction unit 80a (Step S3-1-1). Then, a first aliquot of the specimen (sample) is dispensed in the first cuvette 8 (Step S3-1-2). Further, a reagent R2 (reagent R2 for antibody measurement) including HIV antigen stabilization magnetic particles is dispensed in the first cuvette 8 (Step S3-1-3).

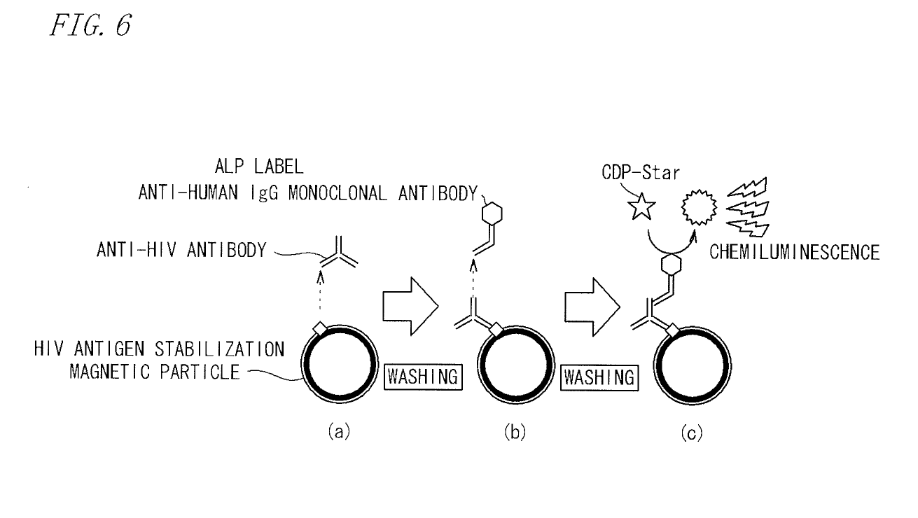

Thus, in a case where an anti-HIV antibody is included in the first aliquot of the specimen, the HIV antibody and the HIV antigen stabilization magnetic particles in the reagent R2 specifically reacts with each other (ref. to FIG. 6(a)).

Subsequently, B/F separation (washing) is carried out by the first B/F separation unit 100a (Step S3-1-4).

Further, a reagent R3 (reagent R3 for antibody measurement) including ALP label anti-IgG antibody monoclonal antibody (mouse) is dispensed in the first cuvette 8 transported to the second reaction unit 80b (Step S3-1-5). Thus, ALP label anti-human IgG monoclonal antibody (mouse) in the reagent R3 specifically reacts with anti-HIV antibody (ref. to FIG. 6(b)).

Then, after the B/F separation (washing) is thus carried out by the second B/F separation unit 100b, a reagent R4 (magnetic particle dispersion solution) is dispensed in the first cuvette 8 (Step S3-1-7) and a reagent R5 including luminescence substrate CDP-Star (produced by Applied Biosystems Inc.) is dispensed in the first cuvette 8 (Step S3-1-8). Then, the luminescence substrate CDP-Star is degraded by ALP (alkaline phosphatase) on the magnetic particle and chemiluminescence is generated (ref. to FIG. 6(c)).

This luminescence is measured by the detection unit 120 (Step S3-1-9).

Thus measured chemiluminescence amount is memorized by the memory of the measurement control unit 140 (Step 3-1-10).

### [Step S3-2: HIV Antigen Measurement Process]

A HIV antigen measurement process according to the present embodiment is purposed to measure HIV-1 p24 antigen in the second aliquot of the specimen.

In the antigen measurement process, as shown in FIG. 5(b), first a reagent R1 (reagent R1 for antigen measurement) including ALP label anti-HIV-1 p24 monoclonal antibody (human) is dispensed in the second cuvette 8 held by the first reaction unit 80a (Step S3-2-1).

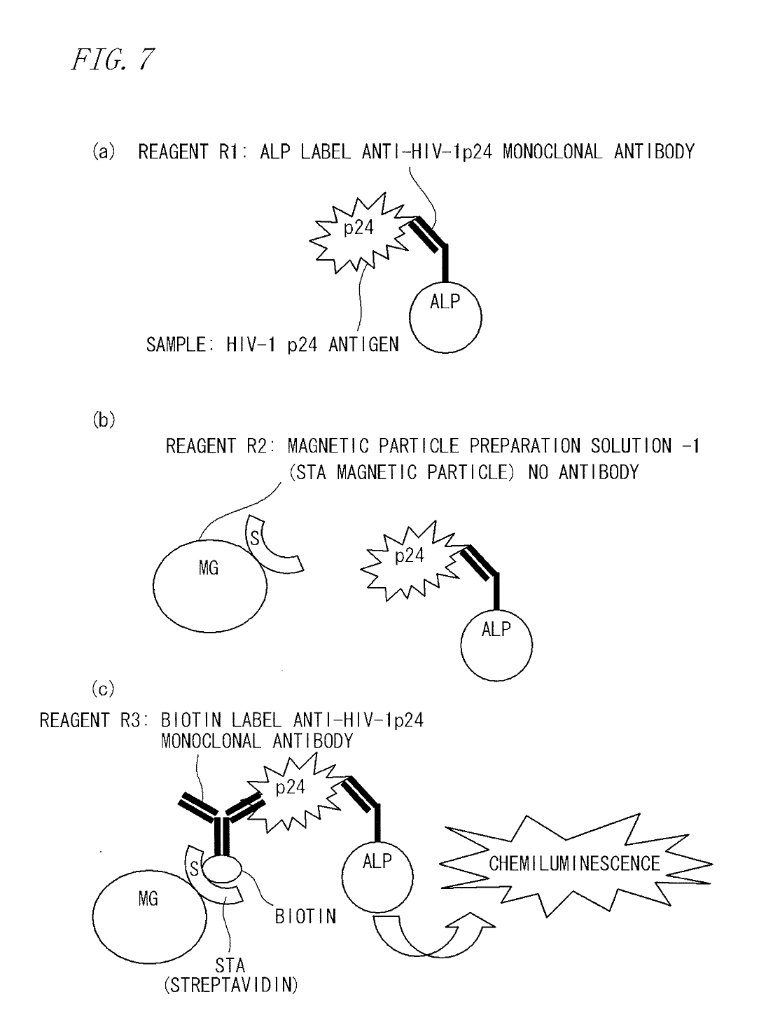

Then, the second aliquot of the specimen (sample) is dispensed in the second cuvette 8 (Step S3-2-2) and reaction is performed for a predetermined time (e.g. 2 minutes 30 seconds). In a case where the HIV-1 p24 antigen is included in the second aliquot of the specimen, the p24 antigen and the ALP label anti-HIV-1 p24 monoclonal antibody (human) specifically reacts with each other (ref. to FIG. 7(a)).

Further, a reagent R2 for antigen measurement (magnetic particle preparation solution-1 (STA (streptavidin) magnetic particle) no antibody) is dispensed in the second cuvette 8 (Step S3-2-3). There is no reaction occurred here (ref. to FIG. 7(b)).

Then, the second cuvette 8 is transported to the second reaction unit 80b through the first B/F separation unit 100a and a reagent R3 including biotin label anti-HIV-1 p24 monoclonal antibody (human) is dispensed in the second cuvette 8 (Step S3-3-4), and reaction is performed for a predetermined time (e.g. 2 minutes 30 seconds). Here, the biotin and the STA reacts with each other and a complex including p24 antigen and the anti-HIV-1 p24 monoclonal antibody (human) reacts with each other.

Subsequently, B/F separation (washing) is carried out (three times) by the second B/F separation unit 100b (Step S3-2-5).

Subsequently, a reagent R4 (magnetic particle dispersion solution) and a reagent R5 including luminescence substrate CDP-Star are dispensed in the second cuvette (Step S3-2-6, Step S3-2-7). Thus, the luminescence substrate CDP-Star is degraded by the ALP (alkaline phosphatase) included in the complex and chemiluminescence is generated (ref. FIG. 7(c)).

This chemiluminescence is measured by the detection unit 120 (Step S3-2-8).

Thus measured chemiluminescence amount is memorized by the memory of the measurement control unit 140 in combination with chemiluminescnece amount indicative of antibody amount with respect to the same specimen (Step S3-2-9).

### [Analysis Process (Step S6)]

In an analysis process, the control device 400 integratedly carries out determination on presence of HIV infection (positive/negative) and others based on chemiluminescence amount (measurement result indicating antibody amount or antigen amount) received from the measurement control unit 140. A HIV antibody measurement result and a HIV antigen measurement result are integrated to determine presence of HIV infection and the determination result is outputted. Therefore, it is easy for the device user to determine presence of HIV infection.

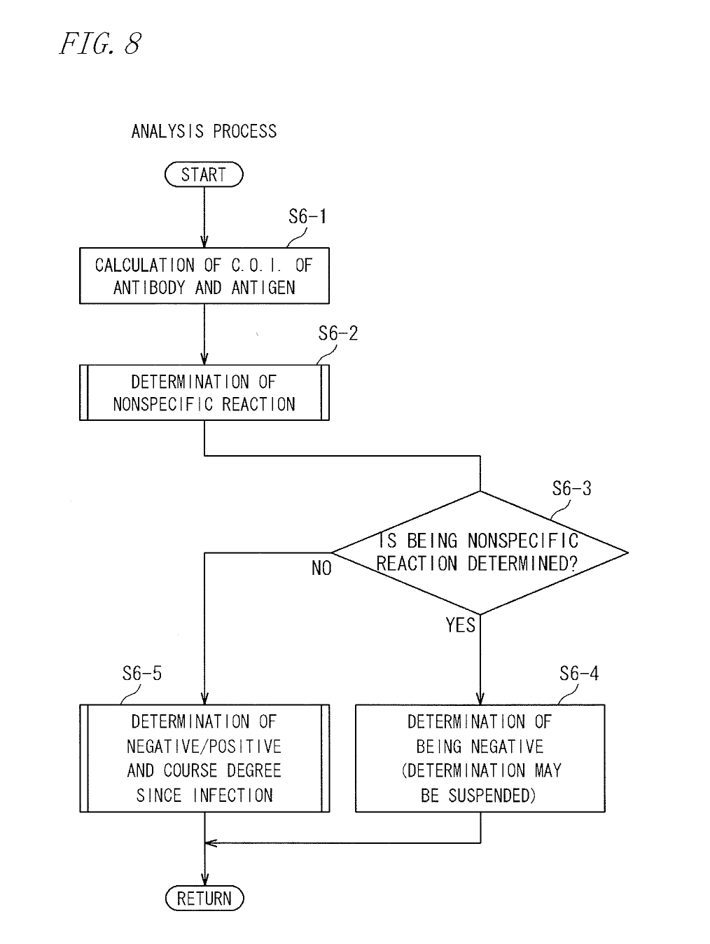

In the analysis process, first, C.O.I. (cutoff index) of antibody and antigen each is calculated, as shown in FIG. 8, (Step S6-1). The C.O.I. indicates an amount of antibody or antigen and becomes an index for determining positive or negative. The C.O.I. is obtained by the following formula.

C.O.I.=(specimen chemiluminescence amount−negative control chemiluminescence amount)/(cutoff control chemiluminescence amount−negative control chemiluminescence amount)

Here, the negative control is a reference material which is prepared so as to make determination result negative, and the cutoff control is a reference material which is prepared so as to make a determination result intermediate between positive and negative.

In a case where C.O.I. is 1 or more, the specimen is usually determined as positive. In a case where C.O.I. is less than 1, the specimen is determined as negative.

In other words, in the immunoassay apparatus according to the present embodiment, in a case where antibody C.O.I. is 1 or more, it is determined as antibody positive and in a case of less than 1, it is determined as antibody negative (antibody determination means). Further, in a case where antigen C.O.I. is 1 or more, it is determined as antigen positive, and in a case of less than 1, it is determined as antigen negative (antigen determination means).

In the analysis process, positive/negative determination is not made with respect to HIV infection immediately after calculation of respective C.O.I. of antibody and C.O.I. of antigen. Determination is made on whether or not the nonspecific reaction occurs, in other words, whether or not there is potential false-positive status (detection of false-positive) (Step S6-2). The determination in Step S6-2 is described later in detail.

When a nonspecific reaction (false-positive) is determined (Step S6-3), negativity (no HIV infection) is determined as a determination result for HIV infection regardless of respective C.O.I. values of antibody and antigen (Step S6-4). Here, in a case where the nonspecific reaction (false-positive) is determined, determination on HIV infection may be suspended.

In a case where the nonspecific reaction (false positive) is not determined, determination is made with respect to presence of HIV infection (negative/positive determination) and a course degree since HIV infection (clinic stage) (Step S6-5). The determination in Step S6-5 is also described later in detail.

### [Relation Between Clinical Stage (Pathological State) an Antibody Amount and Antigen Amount]

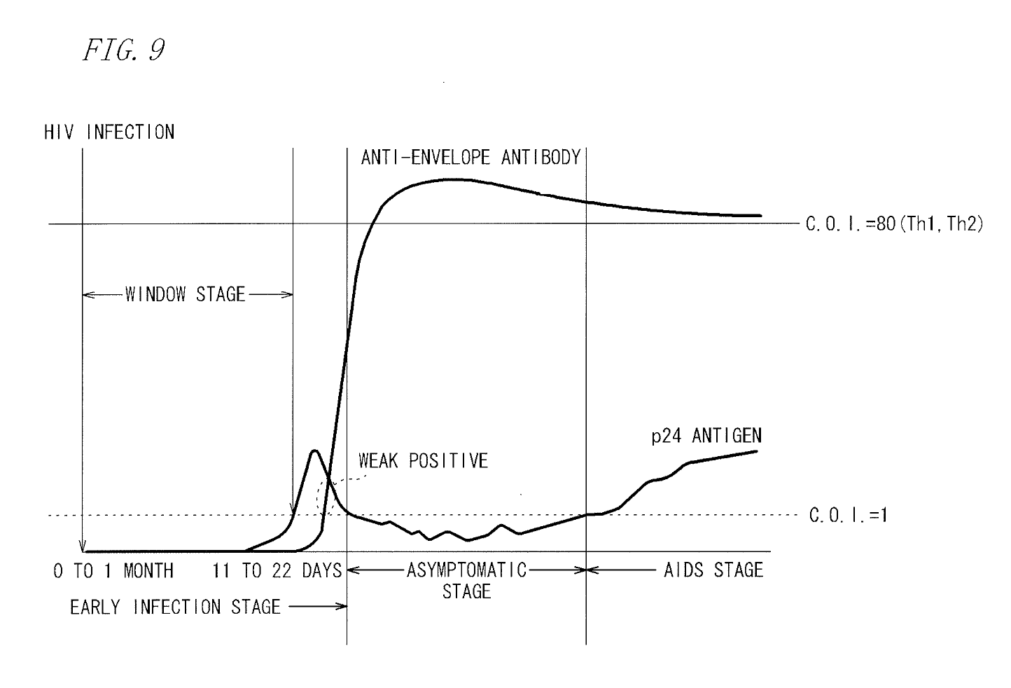

In the present embodiment, time lapse changes of the antibody amount and the antigen amount as shown in FIG. 9 is utilized for analyzing HIV infection based on the antibody (anti-envelop antibody) amount and the antigen (p24 antigen) amount.

As shown in FIG. 9, a pathological state (clinical stage) of a HIV infected person is divided into three stages by time since HIV infection: early infection stage, asymptomatic stage following the early infection stage, AIDS stage following the asymptomatic stage.

The early infection stage is a stage where an influenza-like symptom or a viremia symptom is found, the asymptomatic stage is a stage where any clinical symptom is not found at all, and the AIDS stage is a stage where at least one infection disease among opportunistic infection diseases being a criterion for AIDS is developed.

Immediately after HIV infection, there exists a window stage where the result becomes negative in antibody measurement or antigen measurement because amount of the antibody and the antigen is small during the early infection stage. For this reason, HIV infection cannot be detected in the antibody measurement or the antigen measurement during the window stage.

After the window stage, the HIV antigen begins to increase first and later the HIV antibody also increases along with increase of the HIV antigen.

In the early infection stage, where the HIV antibody begins to increase, the HIV antigen decreases and the HIV antigen amount decreases down to a level (antigen C.O.I.<1) indicative of negative status.

In the present embodiment, a stage where the HIV antigen amount increases once and subsequently decreases immediately before the level of antigen negative (antigen C.O.I.<1) is defined as “early infection stage”. In the early infection stage, usually the antibody just begins to increase and an amount of the antibody is small.

In the present embodiment, a stage where a status is antigen negative while the HIV antibody increases and antibody positive is indicated is defined as “asymptomatic stage”.

After the asymptomatic stage, when an immune apparatus of the subject is broken, the antigen amount increases again and antigen positive comes to be indicated (antigen C.O.I.≧1). In the present embodiment, this stage is defined as “AIDS stage”.

Although the AIDS stage may be also defined as a stage where both of the antibody and the antigen are positive, both of the antibody and the antigen become positive also in the early infection stage in some cases.

Therefore, in the present embodiment, in order to differentiate between the AIDS stage and the early infection stage, a stage where the antigen is positive and the antibody C.O.I. is small (less than the first threshold value Th1; including negative) is defined as an early infection stage. A stage where the antigen is positive and C.O.I. amount of the antibody is large (more than the second threshold value Th2) is defined as an AIDS stage.

Although two threshold values Th1 and Th2 may be same or different, Th1=Th2 is set here for simplification. Also, Th1=Th2=80 is specified.

Therefore, in the present embodiment, three clinical stages (early infection stage, asymptomatic stage, and AIDS stage) of the HIV infection person are defined as follows.

a) Early infection stage: antibody C.O.I.<80 and antigen positive (C.O.I.≧1)
b) Asymptomatic stage: antibody positive (C.O.I.≧1) and antigen negative (C.O.I.<1)
c) AIDS stage: antibody C.O.I.≧80 and antigen positive (C.O.I.≧1)

Here, the above definition of clinical stage in the present embodiment is only an example. Any criterion may be employed, provided that it can be a criterion for specifying course degrees since infection (clinical stage). Particularly, the threshold values Th1 and Th2 are not limited to 80.

The HIV antibody begins to increase later than the HIV antigen as shown in FIG. 9. However, once the HIV antibody increases and becomes positive, it constantly indicates positive since then, so that a HIV antibody measurement result is easy to be used as an index for determining presence of HIV infection.

On the other hand, since the HIV antigen measurement result indicates negative in the asymptomatic stage, HIV antigen alone is difficult to be used as an index for determining presence of HIV infection. However, the HIV antigen begins to increase earlier than the HIV antibody. Therefore, it is possible to shorten the window stage in the early infection stage by performing HIV antigen measurement in addition to HIV antibody measurement.

In other words, basically a status may be concluded as “positive” in HIV infection determination as long as a result of HIV antibody measurement and HIV antigen measurement is HIV antibody positive (antibody C.O. I.≧1) or HIV antigen positive (antigen C.O.I.≧1). In a case where both of the antibody and the antigen are positive, it may be determined as “positive” in HIV infection determination as a matter of course.

As described above, the present inventors found it possible that a course degree since HIV infection is specified by a HIV antibody amount and a HIV antigen amount.

In other words, by defining three clinical stages of the HIV infected person as described above, it is possible not only to determine HIV infection but also to specify a course degree (clinical stage) since HIV infection, provided the antibody amount and antigen amount (C.O.I.) are known.

In the immunoassay apparatus according to the present embodiment, a course degree (clinical stage) since HIV infection can be analyzed based on a HIV antibody amount and a HIV antigen amount, so that it is possible to determine a course degree since HIV infection.

Recognizing the clinical stage is very useful for doctors because it is helpful for a decision on treatment and test and others.

In other words, in a case where it is antigen negative while it is antibody positive, the clinical stage is determined in an asymptomatic stage. Further in a case of antigen positive, it is determined in an early infection stage or in AIDS stage. Furthermore, it is possible to differentiate between the early infection stage and the AIDS stage based on an amount of antibody.

Further, the present inventors found that it is possible to determine false positive (antibody false positive) based on amounts of antibody and antigen. In the HIV antibody measurement, there are some cases where it may be erroneously determined as antibody positive due to a nonspecific reaction although it is originally negative (person not infected with HIV). However, in a case of the nonspecific reaction, strong positive is not indicated but relatively-weak positive (weak positive) is indicated.

Provided such weak positiveness of the antibody is applied to FIG. 9, the weak positiveness of the antibody occurs in the early infection stage in a case of the HIV infected person. As shown in FIG. 9, the antigen also indicates positive in the stage where the antibody is weak positive. In other words, there is no possibility that “the antigen becomes negative while the antibody is weak-positive” in a case of the HIV infected person (positive case).

Therefore, even though the antibody indicates positive, it should actually be negative in a case of “antibody weak positive and antigen negative”. Consequently, in a case of “antibody weak positive and antigen negative”, it may be determined as false positive even though the antibody is positive. That means, since false positive is detectable based on measurement results of HIV antibody amount and HIV antigen amount, it is possible to decrease erroneous determination of positive although it is negative.

In an analysis process of the present embodiment, the above described steps of determination (Steps S6-2, S6-5, S6-4) are carried out from the above viewpoint. Hereinafter, these steps of determination are described in detail.

### [Determination on Nonspecific Reaction (False Positive) (Step S6-2)]

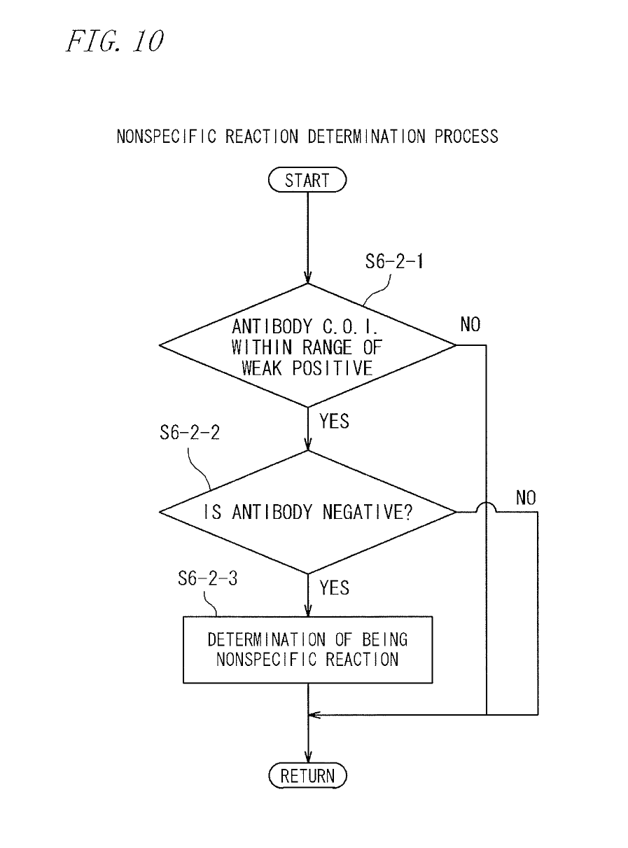

As shown in FIG. 10, it is determined whether or not the antibody amount (C.O.I.) is within a range of weak positive (1≦antibody C.O.I.≦third threshold value) (Step S6-2-1).

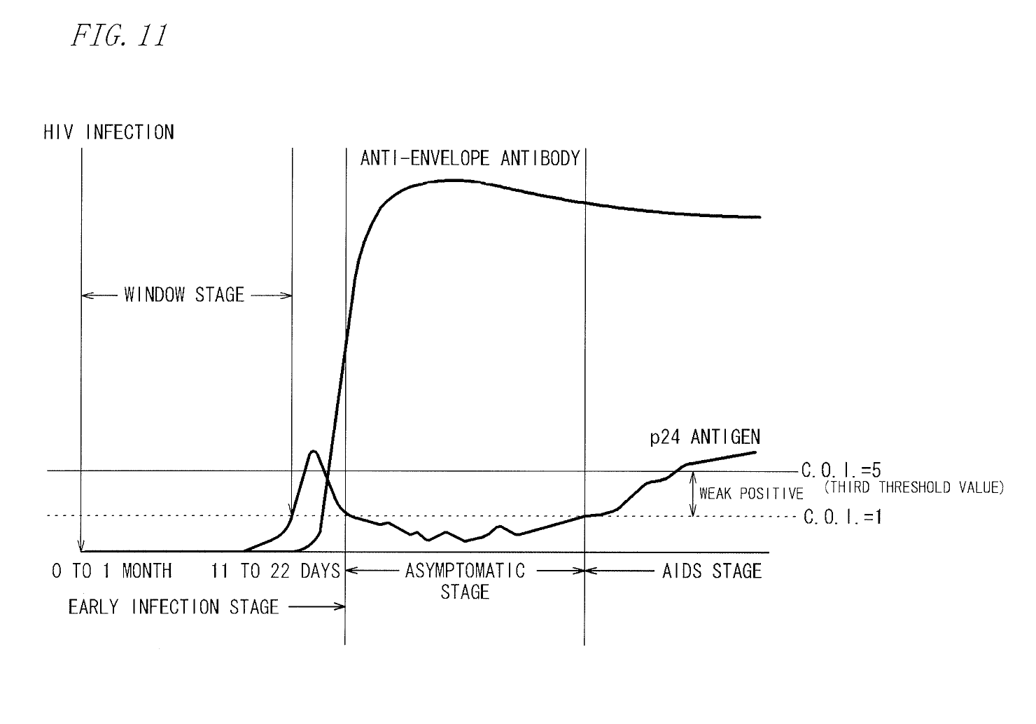

Here, as shown in FIG. 11, the third threshold value is set at C.O.I.=5. However, the third threshold value is not limited to “5” but appropriately settable. For example, the third threshold may be larger depending on the antibody.

In a case where the antibody amount does not indicate weak positive, it is not determined as nonspecific reaction (false positive) and a nonspecific reaction determination process is finished.

In a case where the antibody amount indicates weak positive, it is determined whether or not the antigen is negative, in other words, the antigen C.O.I. is less than 1 (Step S6-2-2). In a case where the antigen is positive, it is not determined as nonspecific reaction (false positive) and the nonspecific reaction determination process is finished.

In a case where the antigen is negative, it is determined as nonspecific reaction (false positive) (Step S6-2-2), and the determination result is stored in a memory and the nonspecific reaction determination process is finished.

When it is determined as nonspecific reaction as a result of the nonspecific reaction determination, it is determined as negative as described before or a determination is suspended (FIG. 8; Step S6-4). This suspension of the determination enables to decrease mental stress for a subject caused by being judged erroneously as positive.

### [Process of Determining Negative/Positive and Course Degree Since Infection (Step S6-5)]

In a case where it is determined as no nonspecific reaction, presence of HIV infection and a course degree since infection (clinical stage) are determined.

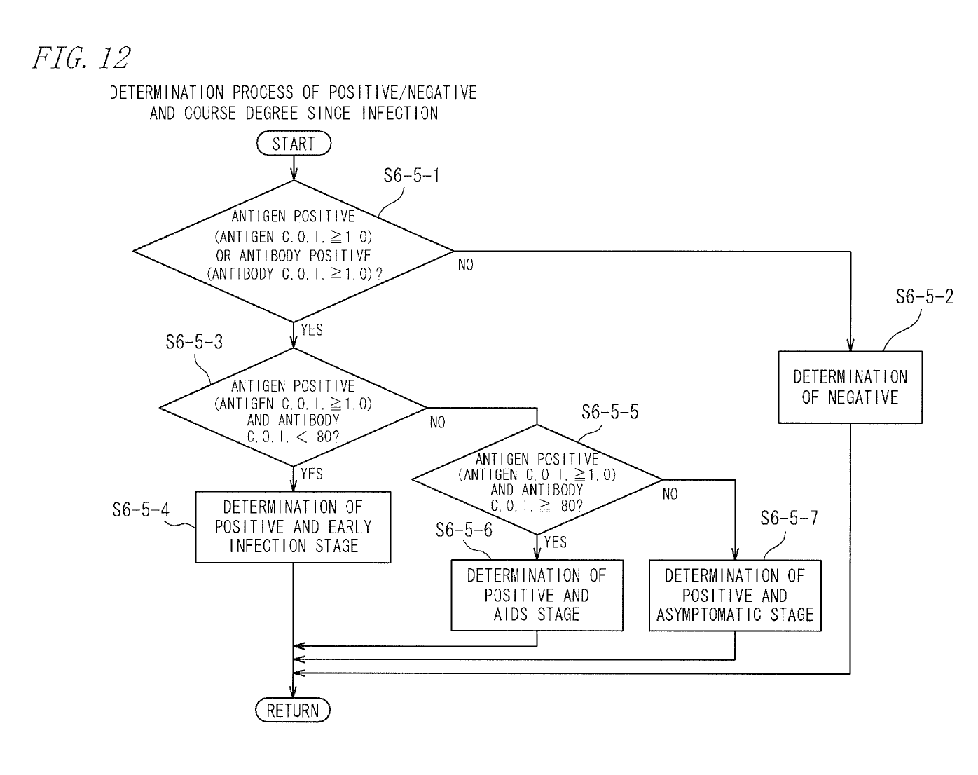

For this, first as shown in FIG. 12, it is determined whether or not at least either one of the antibody and the antigen indicates positive (Step S6-5-1). In a case where both of the antibody and the antigen are negative, the HIV infection determination is resulted as “negative” (Step S6-5-2).

On the other hand, in a case where at least either one of the antibody and the antigen indicates positive, HIV infection determination is resulted as “positive”. However, in order to determine the course degree (clinical stage) since HIV infection, the following process is carried out. Here, since the case of false positive is eliminated, accuracy of the HIV infection determination is high.

First, it is determined whether or not “the antigen is positive and the antibody C.O.I. is less than the first threshold value Th1 (80)” (Step S6-5-3; early infection stage determination). In a case where “the antigen is positive and the antibody C.O.I. is less than the first threshold value Th (80)”, the subject specimen is determined as “positive and in an early infection stage” (Step 56-5-4).

In a case where it is not determined “the antigen is positive and the antibody C.O.I. is less than the first threshold value Th1 (80)” in Step S6-5-3, it is subsequently determined whether or not “the antigen is positive and the antibody C.O.I. is the second threshold value Th2 (80) or more” (Step 56-5-5; AIDS stage determination).

In a case where “the antigen is positive and the antibody C.O.I. is the second threshold value Th2 (80) or more”, the subject specimen is determined as “positive and in an AIDS stage” (Step S6-5-6).

In a case where it is not determined “the antigen is positive and the antibody C.O.I. is the second threshold value Th2 (80) or more” in Step S6-5-5, the subject specimen is determined as “positive and in an asymptomatic stage” since the antibody is positive and the antigen is negative (Step 56-5-7).

As described above, in the present embodiment, divergent judgment included in the computer program is employed for specifying the clinical stage based on the antibody amount and the antigen amount (Steps 56-5-1 to 7). However, a table which correlates the antibody amount and the antigen amount with a corresponding clinical stage may be employed. In other words, the table is referred to by using the antibody amount and the antigen amount which are measured, so that the clinical stage (course degree since infection) may be obtained.

Thus, “information on the correlation between antibody amount/antigen amount and a course degree since infection” for obtaining a course degree since infection based on the antibody amount and the antigen amount may be realized by the divergent judgment included in the computer program (Step S6-5-1 to 7) or realized by the above-described table.

### [Output of Analysis Process Result (Step S7; Output Means)]

When the above analysis process is finished, a determination result is outputted on the display unit 400b as described before.

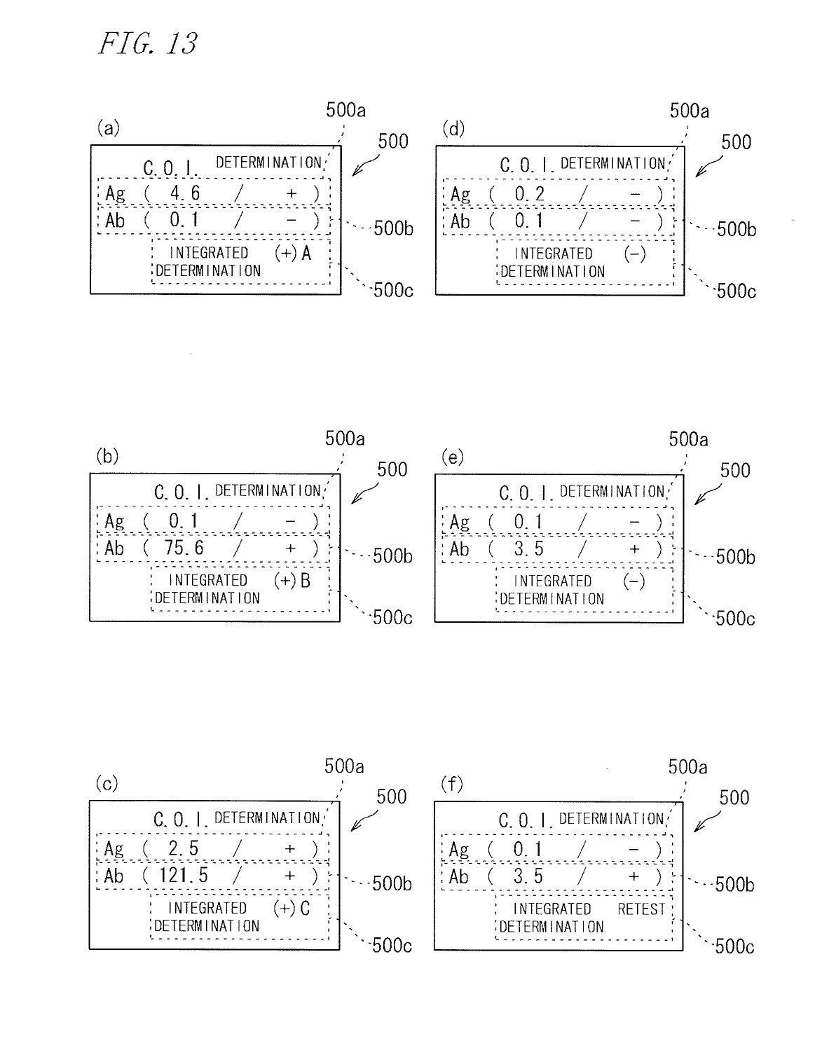

FIGS. 13(a) to 13(f) show an example of the determination result displayed on a determination result display screen 500 of the display unit 400. In the respective display screens 500 of FIGS. 13(a) to 13(f), an antigen result display unit 500a which displays C.O.I. (antigen amount information) and a determination result of positive (+)/negative (−) with respect to the antigen (Ag), an antibody result display unit 500b which displays C.O.I. (antibody amount information) and a determination result of positive (+)/negative (−) with respect to the antibody (Ab), and an integrated determination display unit 500c which displays presence or absence of HIV infection are displayed.

The C.O.I. values (HIV antibody measurement result and HIV antigen measurement result) displayed individually on the antigen result display unit 500a and the antibody result display unit 500b are values calculated in Step S6-1 (ref. to FIG. 8).

Further, the determination result of the antigen result display unit 500a is determination result of antigen C.O.I. alone (antigen positive/antigen negative). In a case where the antigen C.O.I. is 1 or more, “+” (positive) is displayed and in a case of less than 1, “−” (negative) is displayed.

The determination result of the antibody result display unit 500b is determination result of antibody C.O.I. alone (antibody positive/antibody negative). In a case where the antibody C.O.I. is 1 or more, “+” (positive) is displayed and in a case of less than 1, “−” (negative) is displayed.

The integrated determination display unit 500c displays an integrated determination result (presence or absence of HIV infection (positive/negative)) and others and displays a determination result in Step S6-4 or Step S6-5.

In the integrated determination display unit 500c, “+” is displayed in a case where the determination of HIV infection is resulted positive, and “−” is displayed in a case where the determination of HIV infection is resulted negative.

Further the integrated determination display unit 500c displays not only positive/negative but also a course degree since HIV infection (clinical stage).

The clinical stage is indicated by “A” “B” and “C”. “A” indicates an early infection stage, “B” indicates an asymptomatic stage, and “C” indicates an AIDS stage.

Here, FIG. 13(a) shows an example of the display screen 500 where the integrated determination is positive and the clinical stage is the early infection stage (case of Step S6-5-4). Further, FIG. 13(b) shows an example of the display screen 500 where the integrated determination is positive and the clinical stage is the asymptomatic stage (case of Step S6-5-7). FIG. 13(c) shows an example of the display screen 500 where the integrated determination is positive and the clinical stage is the AIDS stage (case of Step S6-5-6).

Further, FIG. 13(d) shows an example of the display screen 500 where the integrated determination is negative (case of Step S6-5-2).

FIGS. 13(e) and 13(f) show examples of the display screen 500 in a case of false positive (case of Step S6-4). In a case of false positive, the integrated determination may be displayed as “−” (negative) as shown in FIG. 13(e) or the determination may be suspended and a message prompting retest is displayed as shown in FIG. 13(f). Further, it is preferable to prompt other test methods such as PCR for retesting. Since a message for prompting the retest is outputted, the retest of the specimen of false positive is ensured to be conducted.

Here, in a case where the determination is suspended, the user knows that the determination is suspended, though determination suspension is not explicitly outputted and only prompting the retest is displayed without display of either positive/negative. Thus it is obvious for the user that the determination is suspended if neither positive nor negative is displayed as a result of HIV infection test.

According to the immunoassay apparatus of the present embodiment, since HIV infection is integratedly determined based on respective measurement results of antibody and antigen, it is possible to provide users (doctors and others) with an accurate determination result on presence of HIV infection in an easy-to-understand manner. Further, in a case of HIV infection, because a course degree since HIV infection (clinical stage) is also displayed, doctors are provided with useful information for determining policies of treatments and tests in response to a course degree.

Besides, in the apparatus of the present embodiment, doctors are provided with useful information for more accurately grasping clinical conditions of the subject such as a course degree since HIV infection, because measurement results or determination results of the antibody and antigen are also displayed. In other words, because a result of the integrated determination is influenced by setting of various threshold values with respect to C.O.I., when measurement results of the antibody and antigen and/or individual determination results based on the measurement results are displayed, it is much useful for doctors to determine the clinical stages and others by themselves.

Further, the measurement results or the determination results of the antibody and the antigen are displayed respectively; this is also useful to the doctors for accumulating experiences with respect to a relation between respective amounts of the antibody and antigen and course degree since HIV infection. Further, it is possible for the apparatus users such as the doctors to assume a course degree since HIV infection based on the respective amounts of the antibody and the antigen (antibody amount information/antigen amount information).

Further, it is easy to determine positive/negative of respective antibody and antigen based on the determination result of HIV antibody and determination result of HIV antigen respectively.

Further, as described above, a confirmation test is easy because positive/negative is determined with respect to the antibody and the antigen respectively.

The confirmation test is a test to manually confirm whether or not the determination result is due to original antigen antibody reaction with respect to a specimen which is determined as positive in the immunoassay apparatus, and an absorbent solution for confirmation including the antibody or the antigen of the subject of measurement is used.

For example, the confirmation absorbent solution for HIV antibody is a solution which includes the HIV antigen in an amount corresponding to a sufficient amount of the HIV antibody included in the specimen. The confirmation absorbent solution for HIV antigen is a solution which includes sufficient amount of HIV antibody included in the specimen.

In order to perform this confirmation test, the specimen is divided again after the test by the immunoassay apparatus, and the specimen is required to be reacted with the confirmation absorbent solution. Here, in a case of antibody positive, the confirmation absorbent solution for antibody is used for the confirmation test, and in a case of antigen positive, the confirmation absorbent solution for antigen is used for the confirmation test.

However, in a case where it is simply known that the specimen is positive but it is not known whether the antibody is positive or the antigen is positive, it is required that the confirmation test is performed using both of the confirmation absorbent solutions for the antibody and for the antigen.

However, according to the immunoassay apparatus of the present embodiment, because positive/negative is determined for the antibody and antigen respectively, it is possible to promptly choose which confirmation absorbent solution should be used and promptly know a result of the confirmation test.

### Measurement Example

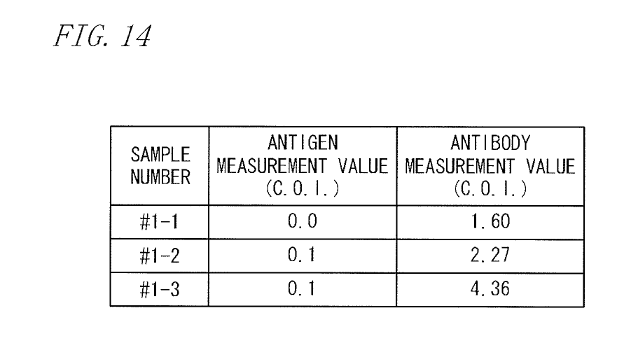

FIGS. 14 to 16 show results of measurement performed on various samples (specimens) by the immunoassay apparatus of the present embodiment.

FIG. 14 shows false positive samples, or samples which are not infected with HIV but the antibody is positive (antibody weak positive) (#1-1 to #1-3). Here, three samples shown in FIG. 14 are taken from different subjects respectively.

As shown in FIG. 14, in a case of false positive, an antibody C.O.I. is in a range of 1 to 5 and the validity that third threshold value for determination of false positive is “5” is supported (ref. to Step S6-2-1). Here, in a case of false positive, the antigen is negative.

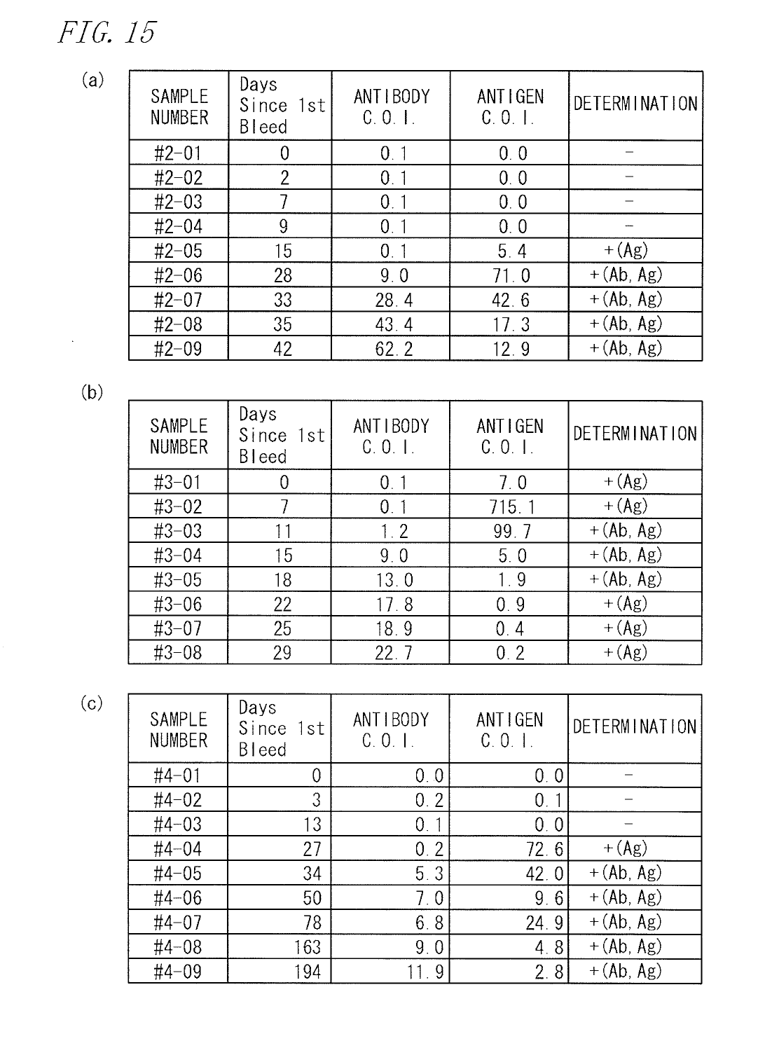

FIG. 15(a) shows a measurement result of samples (#2-01 to #2-09) from a given subject being infected with HIV. In FIG. 15(a), measurement results are listed in the order of blood sampling date with respect to the same subject. In FIG. 15(a), from the first blood sampling date (day 0) to the 9^{th} day, “−” (negative) is shown and it is considered in a window stage of early infection stage. On the 15^{th} day, the antibody is negative and the antigen is positive, and integrated determination is “+” (positive; early infection stage). According to the present embodiment, it is possible for the doctor to find that it is relatively early stage in the early infection stage, because “antigen is positive while antibody is negative” is displayed on the screen.

After the 28^{th} day, all are in an early infection stage, because it is “antibody is positive and antigen is positive” and the antibody C.O.I. is less than 80. Further, after the 33^{rd} day, it is possible for the doctor to grasp that it is relatively close to an asymptomatic stage in early infection stage by observing course information like FIG. 15(a) because the antigen decreases while the antibody increases.

FIG. 15(b) shows measurement results of samples (#3-01 to #3-08) from another subject being infected with HIV. In FIG. 15(b) as well, measurement results are listed in the order of blood sampling date with respect to the same subject.

In FIG. 15(b), on the first blood sampling date (day 0) and the 7^{th} day, “antigen is positive while antibody is negative” and it is in an early infection stage. Further, on the 11^{th} day to the 18^{th} day, “antibody is positive and antigen is positive” and the antibody C.O.I. is less than 80, therefore all are in an early infection stage. Further, on the 22^{nd} day to the 29^{th} day, the antigen is negative while the antibody is positive, therefore it is determined in an asymptomatic stage.

FIG. 15(c) further shows measurement results of the samples (#4-01 to #4-09) from the other subject infected with HIV. In FIG. 15(c) as well, measurement results are listed in the order of blood sampling date with respect to the same subject.

In FIG. 15(c), on the first blood sampling date (day 0) to the 13^{th} day, it is considered in a window stage. On the 27^{th} day to the 194^{th} day, it is determined in an early infection stage.

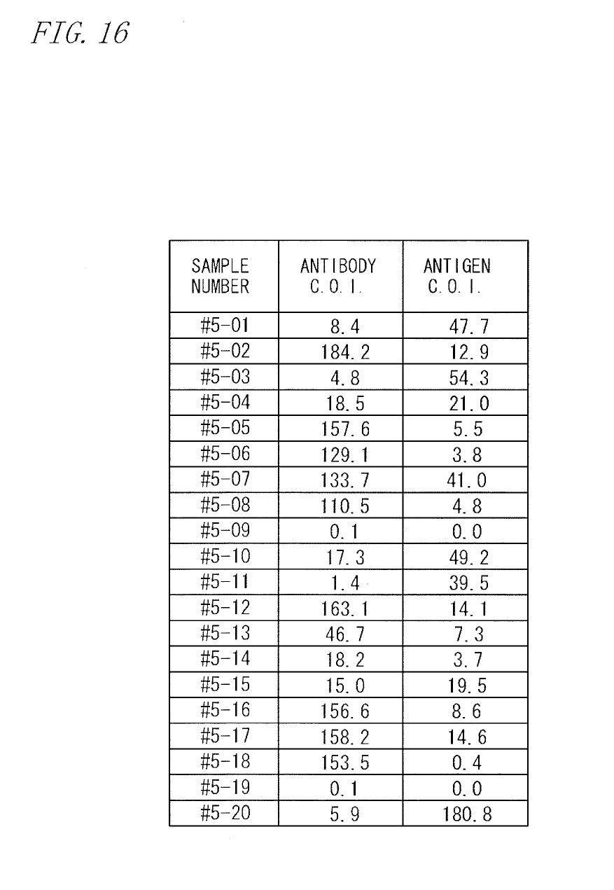

FIG. 16 shows measurement results of the samples (#5-01 to #5-20) from a plurality of subjects different in time lapse since infection.

In a case of the measurement result shown in FIG. 16, according to the apparatus of the present embodiment, samples #5-01, 03, 04, 10, 11, 13, 14, 15 and 20 are determined in an early infection stage. Samples #5-02, 05, 06, 07, 08, 12, 16, 17 and 18 are determined in an AIDS stage. Samples #5-18 is determined in an asymptomatic stage. Samples #5-09, and 19 are determined as negative (not infected with HIV).

Here, the present invention is not limited to the above embodiment but various modifications are available. For example, although the HIV antibody and the HIV antigen are measured in two aliquots respectively in the present embodiment, they may be measured in one aliquot using a reagent for HIV antigen antibody simultaneous measurement.

Further, although the measurements of HIV antibody and HIV antigen are performed in parallel in the present embodiment, the other measurement may be started after one of the measurements is finished.

Further, although negative/positive is not determined with respect to the specimen which has been determined as nonspecific reaction (false positive) in the present embodiment (Step S6-5 is not carried out), the negative/positive may be determined with respect to the specimen which has been determined as nonspecific reaction. In this case, possibility of false positive may be displayed on the display screen 500 while displaying the result of Step S6-5.

Further, although determination on “early infection stage”, “asymptomatic stage”, and “AIDS stage” is conducted in the present embodiment, there is no need to conduct determination on all of these stages but determination may be conducted on any one of them, for example, only early infection stage, only asymptomatic stage, or only AIDS stage. Further, the AIDS stage may be further divided into an early symptom stage and a late symptom stage based on the HIV antigen amount and determination may be conducted. In this case, a threshold value (fourth threshold value) is set for dividing the AIDS stage into the early symptom stage and the late symptom stage. In a case where a HIV antigen amount is not more than the fourth threshold value, it may be determined in the early symptom stage. In a case where it is more than the fourth threshold value, it may be determined in the late symptom stage.

Further, although the antigen C.O.I. with positive/negative (+/−) and the antibody C.O.I. with positive/negative (+/−) are displayed on the display screen 500 in the present embodiment, only C.O.I. may be displayed or only positive/negative (+/−) may be displayed.

Further, although the measurement result of the antigen and antibody is displayed on the same screen with the integrated determination result in the present embodiment, they may be separately displayed.

Further, although the determination result display screen 500 as shown in FIG. 13 is displayed in the present embodiment, a screen 600 for chronologically displaying the determination result of specific subjects may be displayed in addition to the screen 500.

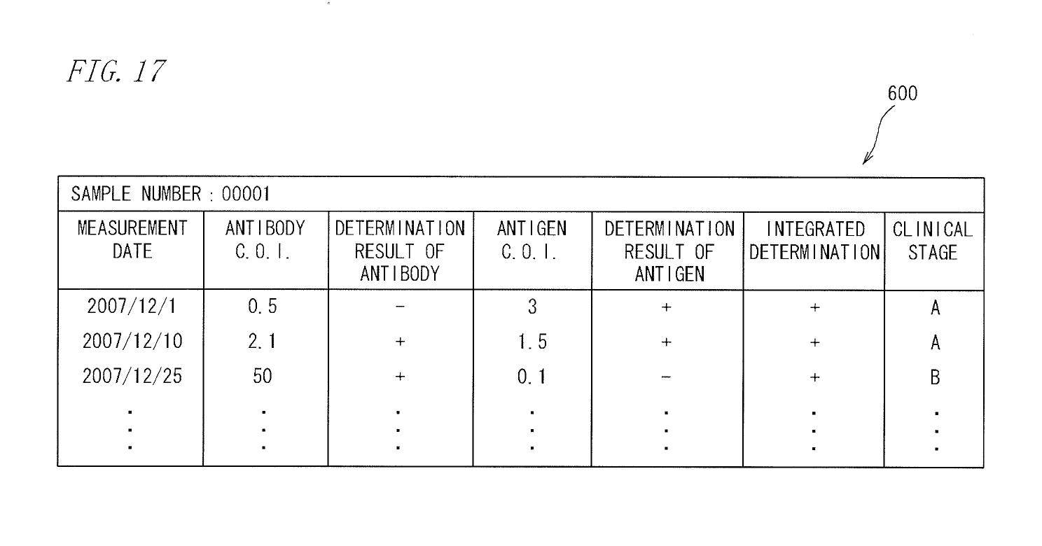

For example, as shown in FIG. 17, the chronological display screen 600 for displaying measurement date, antibody C.O.I., antibody determination result, antigen C.O.I., antigen determination result, integrated determination result and clinical stage may be displayed.

Further, changes of the antibody C.O.I. and the antigen C.O.I. may be displayed as graphs. In said graphs, it is preferable that C.O.I. is represented by a vertical axis and date is represented by a horizontal axis. Such chronological display screen 600 and the graph display are used so that it is easy to confirm change of the measurement results and it is possible to recognize clinical stages (changes of clinical conditions) more assuredly.

Further, although the integrated determination result, the measurement result with determination result of antibody and the measurement result with determination result of the antigen are displayed on the same screen 500 in the present embodiment, these may be displayed on the separate screens.

Further, antibody measurement principle and the antigen measurement principle are not limited to ones disclosed by the present embodiment.

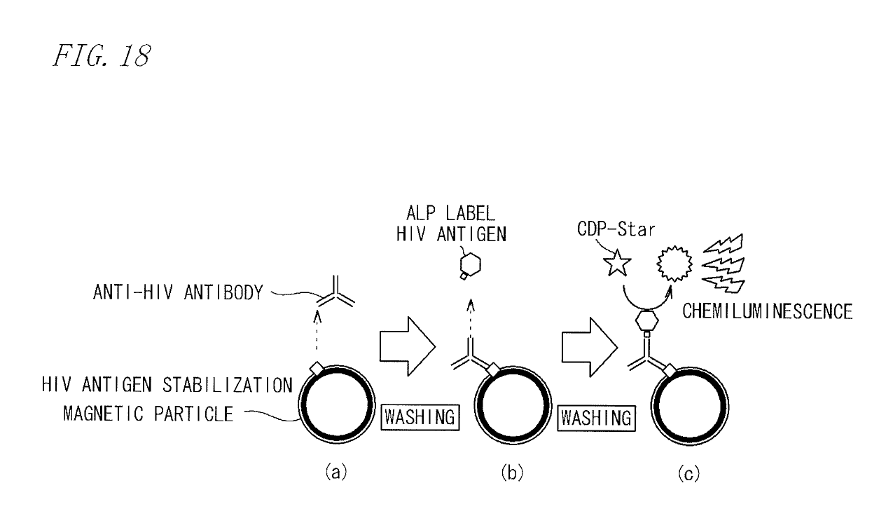

For example, the antibody measurement principle for HIV antibody measurement process in Step S3-1 may be one shown in FIG. 18 instead of one in FIG. 6. In the antibody measurement principle of FIG. 18, ALP label HIV antigen is used instead of ALP label anti-human IgG monoclonal antibody in the antibody measurement principle shown in FIG. 6.

In a case based on the principle of FIG. 18 as well, a flow of antibody measurement process is similar to the flow shown in FIG. 5(a). In other words, first, the R1 reagent for antibody measurement (buffer solution) is dispensed in the first cuvette 8 which is held by the first reaction unit 80a (Step S3-1-1). Then, a first aliquot of a specimen (sample) is dispensed to the first cuvette 8 (Step S3-1-2). Further, R2 reagent including HIV antigen stabilization magnetic particle (R2 reagent for antibody measurement) is dispensed to the first cuvette 8 (Step S3-1-3).

Thus, when anti-HIV antibody is included in the first aliquot of the specimen, the HIV antibody specifically reacts with the HIV antigen stabilization magnetic particle in the R2 reagent (ref. to FIG. 18(a))

Then, B/F separation (washing) is carried out by the first B/F separation unit 100a (Step S3-1-4).

Subsequently, R3 reagent (R3 reagent for antibody measurement) including ALP label HIV antigen is dispensed to the first cuvette 8 transported to the second reaction unit 80b (Step S3-1-5). Thus, the ALP label HIV antigen in the R3 reagent specifically reacts with the anti-HIV antibody (ref. to FIG. 18(b)).

Subsequently, after B/F separation (washing) is carried out by the second B/F separation unit 100b (Step S3-1-6), R4 reagent (magnetic particle dispersion solution) is dispensed to the first cuvette 8 (Step S3-1-7) and R5 reagent including a luminescent substrate CDP-Star (manufactured by Applied Biosystems Inc.) is dispensed to the first cuvette 8 (Step S3-1-8). Then, the luminescent substrate CDP-Star is degraded by ALP (alkaline phosphatase) on the magnetic particle, so that chemiluminescence occurs (ref. to FIG. 18(c)).

The luminescence is measured by the detection unit 120 (Step S3-1-9).

Thus measured chemiluminescence is memorized by the memory of the measurement control unit 140 (Step S3-1-10).

1. An immunoassay apparatus comprising:
a measurement unit for measuring a HIV antibody amount and a HIV antigen amount in a specimen; and
an analysis means for analyzing on HIV infection based on a measurement result which is outputted from the measurement unit, wherein
the analysis means includes a false-positive detection means for detecting false-positive as a result of a HIV infection test, and
the false-positive detection means detects false-positive when the HIV antibody amount is within a range of weak-positive and the HIV antigen amount is within a range of negative.

2. The immunoassay apparatus according to claim 1 further comprising an output means for producing an output indicating that determination of the HIV infection test is suspended or that the HIV infection test is resulted as negative in a case where the false-positive is detected.

3. The immunoassay apparatus according to claim 1, wherein
the measurement unit includes an antibody measurement means for measuring the HIV antibody amount in a first aliquot of the specimen, and
an antigen measurement means for measuring the HIV antigen amount in a second aliquot of the specimen.

4. The immunoassay apparatus according to claim 1, wherein the measurement unit measures the HIV antibody amount and the HIV antigen amount in parallel.

5. The immunoassay apparatus according to claim 1, wherein the measurement unit measures the HIV antibody amount and the HIV antigen amount by measuring a chemiluminescence amount of the specimen.

6. The immunoassay apparatus according to claim 1 further comprising a message output means for outputting a message prompting a retest in a case where false-positive is detected.

7. The immunoassay apparatus according to claim 1, wherein the analysis means analyzes a presence or absence of HIV infection and a course degree since HIV infection.

8. The immunoassay apparatus according to claim 1, wherein the HIV antigen is an antigen HIV-1 p24.

9. The immunoassay apparatus according to claim 1, wherein the HIV antibody is anti-envelope antibody, anti-core antibody, or anti-polymerase antibody.

10. The immunoassay apparatus according to claim 1, wherein the HIV antibody is IgG antibody, IgA antibody, or IgM antibody.

11. An immunoassay method comprising:
an antibody measurement step of measuring a HIV antibody amount in a specimen;
an antigen measurement step of measuring a HIV antigen amount in the specimen; and
an analysis step of analyzing on HIV infection based on a measurement result which is obtained in the antibody measurement step, wherein
the analysis step includes a false-positive detection step of detecting false-positive as a result of a HIV infection test; In the false-positive detection step, false-positive is detected when the HIV antibody amount is within a range of weak-positive and the HIV antigen amount is within a range of negative.

12. The immunoassay method according to claim 11 further comprising an output step of producing an output indicating that determination of the HIV infection test is suspended or that the HIV infection test is resulted as negative in a case where the false-positive is detected.

13. The immunoassay method according to claim 11 further comprising a first aliquot obtaining step of obtaining a first aliquot from the specimen and a second aliquot obtaining step of obtaining a second aliquot from the specimen, wherein a HIV antibody amount in the first aliquot is measured in the antibody measurement step, and a HIV antigen amount in the second aliquot is measured in the antigen measurement step.

14. An immunoassay apparatus comprising:
a measurement unit for measuring a HIV antibody amount and a HIV antigen amount in a specimen; and
an analysis unit for analyzing on HIV infection based on a measurement result which is outputted from the measurement unit and detecting false-positive as a result of a HIV infection test when the HIV antibody amount is within a range of weak-positive and the HIV antigen amount is within a range of negative.

15. The immunoassay apparatus according to claim 14 further comprising a display to produce an output indicating that determination of HIV infection test is suspended or that HIV infection test is resulted as negative in a case where the false-positive is detected.

16. An immunoassay apparatus comprising:
a measurement unit for measuring a HIV antibody amount and a HIV antigen amount in a specimen; and
an analysis unit for analyzing a course degree since HIV infection based on a measurement result outputted from the measurement unit.

17. The immunoassay apparatus according to claim 16 wherein the analysis unit determines whether or not the specimen is one in an early infection stage based on the measurement result outputted from the measurement unit.

18. The immunoassay apparatus according to claim 16 wherein the analysis unit determines whether or not the specimen is one in an AIDS stage based on the measurement result outputted from the measurement unit.

19. The immunoassay apparatus according to claim 16 wherein the analysis unit determines whether the specimen is one in the early infection stage, in an asymptomatic stage following the early infection stage, or in the AIDS stage following the asymptomatic stage, based on the measurement result outputted from the measurement unit.

20. The immunoassay apparatus according to claim 16 wherein the analysis unit determines whether or not the specimen is positive based on the measurement result outputted from the measurement unit and analyzes a course degree since HIV infection in a case where it is determined as positive.

21. The immunoassay apparatus according to claim 16 wherein the analysis unit determines that the specimen is one in the early infection stage when a HIV antigen amount measured by the measurement unit is an amount indicative of positive and a HIV antibody amount measured by the measurement unit is less than a first threshold value.

22. The immunoassay apparatus according to claim 16 wherein the analysis unit determines that the specimen is one in the AIDS stage when a HIV antigen amount measured by the measurement unit is an amount indicative of positive and the HIV antibody amount measured by the measurement unit is an amount indicative of positive and is larger than a second threshold value.

23. The immunoassay apparatus according to claim 16 wherein the analysis unit determines that the specimen is in the asymptomatic stage when a HIV antigen amount measured by the measurement unit is an amount indicative of negative and the HIV antibody amount measured by the measurement unit is an amount indicative of positive.

24. An immunoassay apparatus comprising:
a measurement unit for measuring a HIV antibody and a HIV antigen which are included in a specimen;
an antibody analysis means for analyzing a HIV antibody measurement result by the measurement unit;
an antigen analysis means for analyzing a HIV antigen measurement result by the measurement unit;
an infection determination means for determining presence or absence of HIV infection based on integration of the HIV antibody measurement result and the HIV antigen measurement result which are measured by the measurement unit; and
an output means for outputting the determination result made by the infection determination means, the analysis result made by the antibody analysis means, and the analysis result made by the antigen analysis means.

25. The immunoassay apparatus according to claim 24, wherein the antibody analysis means generates antibody amount information indicative of the HIV antibody amount, the antigen analysis means generates antigen amount information indicative of the HIV antigen amount, and the output means outputs the antibody amount information as an analysis result by the antibody analysis means and outputs the antigen amount information as an analysis result by the antigen analysis means.

26. The immunoassay apparatus according to claim 24, wherein the antibody analysis means determines whether the HIV antibody is positive or negative based on the HIV antibody measurement result by the measurement unit, the antigen analysis means determines whether the HIV antigen is positive or negative based on the HIV antigen measurement result by the measurement unit, and the output means outputs a HIV antibody determination result as an analysis result by the antibody analysis means and outputs the HIV antigen determination result as an analysis result by the antigen analysis means.

27. The immunoassay apparatus according to claim 26, wherein the infection determination means determines presence of HIV infection using the HIV antibody determination result and the HIV antigen determination result.

28. The immunoassay apparatus according to claim 24, wherein the infection determination means determines that HIV infection presents in a case where the HIV antibody measurement result by the measurement unit indicates HIV antibody positive and/or the HIV antigen measurement result by the measurement unit indicates HIV antigen positive.

29. The immunoassay apparatus according to claim 24 further comprising a transportation device for transporting the specimen container to a suction position for the measurement unit to suck in the specimen.

30. The immunoassay apparatus according to claim 24, wherein the output means chronologically displays a determination result by the infection determination means, an analysis result by the antibody analysis means, and an analysis result by the antigen analysis means with respect to a specific subject.

31. An immunoassay apparatus comprising:
a measurement unit for measuring a HIV antibody and a HIV antigen which are included in a specimen;
an analysis unit for analyzing a HIV antibody measurement result by the measurement unit, analyzing a HIV antigen measurement result by the measurement unit, and determining presence of HIV infection based on integration of the HIV antibody measurement result and the HIV antigen measurement result which are measured by the measurement unit; and
a display for outputting an analysis result of the HIV antigen measurement result, an analysis result of the HIV antibody measurement result, and a HIV infection determination result.
